(19) United States
(12) Patent Application Publication
(10) Pub. No.: US 2004/0131690 A1
(43) Pub. Date: Jul. 8, 2004

(54) Beads and methods of preparation

(76) Inventor: Maurice Gerard Lynch, Waterloo (BE)

Correspondence Address: Patent Administrator, FMC Corporation, 1735 Market Street, Philadelphia, PA, 19103, US

(21) Appl. No.: 10/665,811

(22) Filed: Sep. 18, 2003

Related U.S. Application Data
(60) Provisional application No. 60/412,427, filed on Sep. 20, 2002.

(51) Int. Cl. A61K 9/14

(52) U.S. Cl. 424/489; 106/163.01

(57) ABSTRACT

The present invention is directed to beads comprising a carrier liquid and flocculated microcrystalline cellulose, processes for making such beads and to various compositions, such as, cosmetic, pharmaceutical, neutraceutical, veterinary or food compositions, containing such beads.

This application claims the benefit U.S. Provisional Application No. 60/412,427, filed Sep. 20, 2002.

The present invention relates to beads comprising a carrier liquid and flocculated microcrystalline cellulose, as well as methods for the preparation and use thereof. These beads are suitable for applications including cosmetic, pharmaceutical, nutraceutical, veterinary and food applications.

### BACKGROUND OF THE INVENTION

Beads suitable for use in cosmetic applications (1) break down completely and leave no residue as a result of gentle rubbing on the skin and (2) are stable to storage. We have unexpectedly discovered that suitable beads can be prepared comprising microcrystalline cellulose. The beads of this invention break down completely upon rubbing on the skin to release the entrapped materials. These beads provide an excellent skin feel and a strong visual impact and are stable to storage.

### SUMMARY OF THE INVENTION

The present invention is directed to beads comprising a liquid carrier and microcrystalline cellulose. The texture of the beads may be modified by incorporation of an optional texture modifier.

In one embodiment, beads of the present invention are sufficiently robust to be handled and stored in a liquid such as water or brine or a pharmaceutical or cosmetic base without leakage of the internal contents and yet readily release their internal contents when subjected to a physical force. In a preferred embodiment, the beads release their internal contents when ruptured by finger pressure and disintegrate upon gentle rubbing such that no bead residue is detected by sight or touch.

The present invention is also directed to a process for making the beads of the invention comprising the steps of a) dispersing colloidal microcrystalline cellulose in a starting liquid and b) adding the starting liquid into a setting bath to form beads.

One embodiment of the process of the invention is directed towards a process for manufacturing such beads comprising the steps of (a) preparing an aqueous dispersion comprising colloidal microcrystalline cellulose in water; and (b) adding portions of the dispersion to a setting bath comprising water and at least one flocculating agent selected from a salt, an organic solvent, a pH modifier or a cationic material thereby forming beads of sufficient strength to permit recovery of the beads. In further embodiments, a texture modifier may be incorporated in the starting liquid.

In another embodiment, the beads are coated with a material that modifies their texture, stability or release characteristics, for example, by stirring the beads in a bath containing a coating material, for example, chitosan.

In yet another embodiment, the invention is directed towards a process for the manufacture of oil-filled beads comprising the steps of (a) preparing an aqueous dispersion comprising colloidal microcrystalline cellulose; (b) preparing an oil phase comprising an oil; (c) combining the aqueous dispersion and the oil phase to form an emulsion; and (d) adding portions of the emulsion to a setting bath comprising water and at least one flocculating agent selected from a salt, an organic solvent, a pH modifier or a cationic material, thereby forming beads of sufficient strength to permit recovery of the beads. In further embodiments, a texture modifier may be incorporated in the aqueous dispersion or in the oil phase.

Beads of the present invention can be produced in a variety of shapes and sizes and can be used in a number of applications, for example, cosmetic, pharmaceutical, nutraceutical, veterinary, food and other applications.

### DETAILED DESCRIPTION OF THE INVENTION

The term beads, as used herein, refers to small solid or semi-solid objects with diameters generally less than about 5000 microns, preferably from about 25 to about 2500 microns. The beads of this invention can be oval, spherical, oblong or an irregular shape. The beads of this invention are typically soft, that is, they can be ruptured by applying a force such as the force achieved by squeezing a bead between the thumb and index figure.

The beads of the present invention comprise a liquid carrier and microcrystalline cellulose. The liquid carrier comprises water. The beads are manufactured by (a) preparing a starting liquid and (b) adding the starting liquid to a setting bath comprising water and at least one flocculating agent selected from a setting salt, an organic solvent, pH modifier and a cationic material. In one embodiment, the starting liquid is an aqueous dispersion comprising water and colloidal microcrystalline cellulose. The aqueous dispersion is prepared by dispersing colloidal microcrystalline cellulose in water using sufficient shear to achieve substantial dispersion of the microcrystalline cellulose. In another embodiment, the starting liquid is an emulsion prepared by mixing an aqueous dispersion comprising colloidal microcrystalline cellulose and water with an oil phase. The oil phase comprises oil and optionally an emulsifier.

The time interval between adding the starting liquid to the setting bath and recovering the beads should be sufficient to allow the beads to withstand the force involved in recovery. The beads may be washed after removal from the setting bath. Beads may be stored in a suitable aqueous medium that may contain optional stabilizers such as, for example, a flocculating agent or a preservative. The beads of the present invention can be incorporated into aqueous, semi-aqueous or non-aqueous formulations. Those skilled in the art will recognize that the desired storage criteria will vary depending upon the specific end use application.

Microcrystalline cellulose is a purified, partially depolymerized cellulose produced by treating a source of cellulose, preferably alpha cellulose, with a mineral acid. Alpha cellulose contains crystalline regions and paracrystalline regions. The crystalline regions have a high degree of molecular order and the paracrystalline regions are less ordered. Acid selectively hydrolyses the cellulose polymer chain at the paracrystalline regions. The crystalline regions, that constitute microcrystalline cellulose, are then separated from the reaction mixture.

Colloidal microcrystalline cellulose (CMCC) is produced by attriting microcrystalline cellulose to reduce the size of the crystalline regions. Attrited microcrystalline cellulose may be admixed with hydrocolloids and co-dried as taught by Tiemstra in U.S. Pat. No. 3,573,058. The colloidal microcrystalline cellulose may be co-processed with a surfactant as disclosed in Krawczyk in U.S. Pat. No. 6,025,037 or with a binder prior to drying to enable re-dispersion of the colloidal particles, e.g., in U.S. Pat. Nos. 3,539,365 and 5,366,742. The attrition may be in the presence of an attriting aid as disclosed by Venables in U.S. Pat. No. 6,037,080. Microcrystalline cellulose may also be produced by steam explosion as described by Ha in U.S. Pat. No. 5,769,934. Colloidal microcrystalline cellulose has a mean particle size of less than 10 microns, preferably, less than 1 micron. Suitable colloidal microcrystalline celluloses for use in the present invention include colloidal microcrystalline cellulose coprocessed with modified celluloses or with hydrocolloids such as carrageenans and alginates. Examples of coprocessed mixtures include those commercially available from FMC Corporation and sold under the tradenames AVICEL RC-581, AVICEL CL-611 and AVICEL AC-815.

The amount of the colloidal microcrystalline cellulose used to prepare the beads of the present invention is generally from about 0.5 to about 5% by weight of the bead, preferably from about 0.5 to about 2% by weight of the bead.

Colloidal microcrystalline cellulose forms a three dimensional structuring network when dispersed in water. Dispersion is achieved by adding microcrystalline cellulose, which is typically available commercially as a powder, to water and applying sufficient shear to cause separation of individual microcrystals. It is critical to the current invention that the colloidal microcrystalline cellulose be in a substantially dispersed form in the starting liquid. To verify that that the colloidal microcrystalline cellulose is substantially dispersed, a sample of the dispersion can be viewed under a microscope using polarized light and a magnification of 100×. If the microcrystals are substantially dispersed, they will appear as individual white specks homogeneously distributed on a black background.

Although not bound by theory, it is believed that when the starting liquid makes contact with the setting bath, the colloidal microcrystalline cellulose is flocculated by the flocculating agent and forms the structuring matrix of the bead. By flocculation it is meant that the three dimensional network of microcrystalline cellulose collapses due to aggregation of the microcrystals. This is achieved by including at least one flocculating agent in the setting bath. Suitable flocculating agents include setting salts, pH modifiers, organic solvents and cationic materials. Examples of suitable salts include sodium chloride, calcium chloride and potassium chloride. Examples of suitable pH modifiers include citric acid and lactic acid. Examples of suitable organic solvents include ethanol and isopropyl alcohol. Examples of suitable cationic materials include cationic surfactants and cationic polymers.

When present as a flocculating agent, the setting salt is present in the setting bath in an amount of up to 10% by weight of the setting bath, more preferably, from 0.05% by weight to 5% by weight of the setting bath, most preferably from 0.1% to 3% by weight of the setting bath. When present as a flocculating agent, the organic solvent is present in the setting bath at an amount of up to 80%, more preferably from 20% to 70%, most preferably from 20% to 60% by weight of the setting bath.

The temperature of the starting liquid can be from about 5° C. to about 95° C., preferably from about 20° C. to about 95° C. The temperature of the setting bath can be from about 2° C. to about 80° C., preferably from about 2° C. to about 30° C.

Optionally, the beads may contain a texture modifier. The texture modifier modifies properties of the bead such as flexibility, strength, stability, release characteristics, skin-feel and the like. The texture modifier can be used at up to 20% by weight of the total weight of the bead, preferably at up to 2% by weight of the total bead. Examples of suitable texture modifiers include waxes, surfactants, polymers, humectants, and particulate materials. Examples of suitable waxes include beeswax and canuba wax. Examples of suitable surfactants include stearates. Examples of suitable polymers include natural polymers, such as alginates, carrageenans, guar, gelatin, locust bean gum, xanthan and pectin, modified polymers, such as modified celluloses and modified guars, and synthetic polymers such as acrylate-based and vinylpyrolidone-based polymers.

Optionally, the texture modifier may be reactive with the contents of the setting bath, for example with the flocculating agent. Suitable reactive texture modifiers include polymers, for example carrageenan and alginate. For example, potassium or calcium ions are reactive with iota carrageenan and calcium ion is reactive with alginate. Hydrocolloids such as alginate and carrageenan may also be insolubilized and precipitated by organic solvents such as alcohols. Although, not meaning to be bound by theory, it is speculated that the product of the reaction between the texture modifier with the flocculating agent will contribute to the structuring matrix of the bead.

Alternatively, a decrease in the temperature of the starting liquid on addition to a setting bath at a lower temperature can result in a substantial increase in structure. For example, waxes will change from a liquid form to a solid or waxy solid form on cooling and hot solutions of gelling agents, for example gelatin, agar or carrageenan, will change from a liquid form to a gel form on cooling.

Both the starting liquid and the setting bath may optionally contain additional components such as active ingredients, fragrances, flavors, humectants, thickeners, film formers, dyes, pigments, opacificers, pearlescent agents, acids, chelating agents, preservatives and so forth. Active ingredients include ingredients providing a cosmetic, pharmaceutical, nutraceutical, medical or cleansing benefit. Thickeners may include natural polymers, chemically modified polymers or synthetic polymers. When these additional components are added to the starting liquid, they may be added either prior to, concurrent with, or subsequent to addition of the colloidal microcrystalline cellulose to water so long as the microcrystalline cellulose is substantially dispersed in the starting liquid. It is preferred to add the microcrystalline cellulose to water to form an aqueous dispersion of microcrystalline cellulose prior to addition of other components.

Beads may be formed which contain an oil, or combination of oils. Suitable oils include, without limitation, oils derived from mineral sources, animals, plants, microorganisms, or extracts thereof; oils that are chemical compounds derived by synthetic or other means, or formulations thereof; oils that are fatty acids, esters, or derivatives thereof; or oils that may be a pharmaceutically active agent, a nutritional supplement, flavor oil, or a food. Oils suitable for cosmetic applications include for example silicone oils, perfumes, emollients and the like. Oils within the scope of the present invention also include oils that act as carriers or solvents for oil-soluble active materials such as an oil-soluble pharmaceutically active agent, a nutritional, flavor, fragrance, supplement, or a food. Other oils within the scope of the present invention are those that include naturally occurring emulsifiers. Preferred oils within the scope of the present invention are those that are a liquid, or that can be made into a liquid at a temperature in the range of, for example, 20° C. to 95° C. The beads of this invention can contain up to 60% oils.

In one preferred embodiment of the present invention, rupturable beads comprising flocculated colloidal microcrystalline cellulose and optionally a texture modifier are produced which have sufficient strength for handling and storage yet are readily ruptured to deliver their contents by gentle rubbing on the skin. These rupturable beads are suitable for use in, for example, cosmetic or pharmaceutical applications.

Beads of the present invention have a soft pleasant skin-feel, are stable in cream or lotion bases and disintegrate completely when rubbed onto the skin.

In other embodiments, the beads of the present invention can be formed which rupture on the application of internal or external pressure, for example as a result of spraying, squirting, smearing or otherwise removing the beads or compositions containing the beads from a container, or will disintegrate for example when placed in water to deliver their immobilized contents.

In a preferred embodiment, the starting liquid is prepared as follows: an aqueous dispersion is prepared by dispersing colloidal microcrystalline cellulose in water by mixing for five minutes at high speed using a Silverson mixer. An oil phase is prepared by combining a surfactant and oil. The oil mixture and the aqueous base are combined and homogenized at high speed in a Silverson mixer for 2 minutes to form an oil-in-water emulsion. Portions of the dispersion are then deposited into a setting bath comprising 60% by weight of a water brine and 40% by weight of an alcohol in order to form the beads. The beads are removed from the setting bath and rinsed.

When the force applied to the beads exceeds their rupture strength, the beads break and release their immobilized contents.

The advantageous properties of this invention can be observed by reference to the following examples, which illustrate but do not limit the invention.

### EXAMPLES

Materials

In all cases, the water used was deionized water. All percentages are weight by weight.

| Name | Function | Tradename | Supplier |
|---|---|---|---|
| Alginate 1 | Texture modifier | | FMC |
| Alginate 2 | Texture modifier | | FMC |
| Alginate 3 | Texture modifier | | FMC |
| Alginate 4 | Texture modifier | | FMC |
| Betacarotene | Colorant | | Roche |
| (30% dispersion | | | |
| of betacarotene | | | |
| in oil) | | | |
| CMCC | Structuring agent | AVICEL ® CL 611^{a} | FMC |
| CMCC | Structuring agent | AVICEL ® RC-581^{a} | FMC |
| CMCC | Structuring agent | AVICEL ® AC-815^{b} | FMC |
| Carrageenan | Texture modifer | VISCARIN ® SD-389 | FMC |
| Oleth 20 | Surfactant | BRIJ ® 98V | Uniqema |
| C12-15 | Emollient | Finsolv FN | Finetex |
| Alkyl benzoate | | | |
| Titanium | Opacifier | Titanium dioxide, | Aldrich |
| dioxide | | 325 mesh | |
| Mineral Oil | Emollient | KLEAROL | Crompton |
| Silicone oil | Emollient | DC 200 Dimethicone | Dow |
| | | | Corning |
| Sunflower oil | Emollient | Sunflower oil | Vande- |
| | | | moortel |

^{a}Microcrystalline cellulose and sodium carboxymethylcellulose
^{b}Microcrystalline cellulose and alginate

Alginate 1, extracted from the leaves of Laminaria hyperborea, had a viscosity of 50 cP measured at 1% and 20° C. using a Brookfield LV at 20 rpm. Alginate 2, extracted from the leaves of Laminaria hyperborea, had a viscosity of 170 cP measured at 1% and 20° C. using a Brookfield LV at 20 rpm. Alginate 3, a mix of alginate 1 and 2 in the weight ratio of 77 to 23. Alginate 4, extracted from the stems of Laminaria hyperborea, had a viscosity of 300 to 700 cP measured at 10% and 20° C. using a Brookfield LV at 20 rpm.

Procedures

An aqueous dispersion of colloidal microcrystalline cellulose was prepared by slowly adding the colloidal microcrystalline cellulose powder to water while mixing at high speed using a Silverson mixer and continuing to mix for 5 minutes. When alginates or carrageenans were used, they were added after the colloidal microcrystalline cellulose was fully dispersed followed by mixing at high speed for an additional 5 minutes. The dispersion was then heated to 60° C. with stirring slowly with a propeller mixer. An oil phase was prepared separately by combining the oil, surfactant and colorant and heating to 60° C. The aqueous dispersion at 60° C. was added to the oil phase at 60° C. and homogenized at high speed using a Silverson mixer at 60° C. for ten minutes to form a viscous emulsion. The emulsion was maintained at 60° C. while pipetting dropwise into a 1 liter setting bath at 20° C. that was being mixed with a magnetic stirrer. The beads formed were allowed to remain in the bath to harden, typically for about 1 hour. The setting bath compositions that were used in the examples are listed in Table 1. The beads were removed from the setting bath by gently scooping the beads into a dish. The beads were rinsed with deionized water or with a mixture of water and alcohol and were stored in deionized water or in a mixture of water and alcohol. In some cases, the beads were separated from the bath by straining such that the beads were retained on a sieve. The beads were then washed by slowly pouring deionized water over them for about 2 minutes. The washed beads were then transferred to deionized water for storage.

TABLE 1
Setting Bath Compositions (% are weight by weight)

| | Bath A | Bath B | Bath C | Bath D | Bath E |
|---|---|---|---|---|---|
| CaCl2 | 2% | 2% | — | — | 2% |
| KCl | — | — | 2% | 2% | — |
| Ethanol | — | 30% | — | 30% | 40% |
| Water | 98% | 68% | 98% | 68% | 58% |

### Examples 1-1 to 1-4

Starting liquids for beads prepared with different types and levels of colloidal microcrystalline cellulose.

| Components | 1-1 | 1-2 | 1-3 | 1-4 |
|---|---|---|---|---|
| AVICEL ® CL-611 | 2.00% | 1.20% | — | — |
| AVICEL ® RC-581 | — | — | 2.00% | — |
| Silicone oil | 40.00% | — | 40.00% | 40.00% |
| Sunflower oil | — | 55.00% | — | — |
| Surfactant | 3.00% | 4.50% | 3.00% | 3.00% |
| Water | 54.95% | 39.25% | 54.95% | 54.95% |
| Colorant | 0.05% | 0.05% | 0.05% | 0.05% |

Each of the above starting liquids was depositied into both setting baths A and B and the beads formed were removed after about 1 hour and were evaluated. Beads formed by depositing Examples 1-1 and 1-2 into setting baths A and B had a preferred spherical shape while beads formed from Example 1-3 and 1-4 exhibited slight tailing. The strength of beads formed from Example 1-4 was higher than the other beads. On storage of bead of prepared from Example 1-4 for 1 month at room temperature in deionized water, the beads remained separate and did not agglomerate.

### Examples 2-1 to 2-5

Starting liquids for beads prepared with different levels of colloidal microcrystalline cellulose and alginate.

| Components | 2-1 | 2-2 | 2-3 | 2-4 | 2-5 |
|---|---|---|---|---|---|
| AVICEL ® CL-611 | 1.20% | 1.20% | 1.80% | 1.20% | — |
| Alginate 3 | 0.20% | 0.40% | 0.20% | 0.20% | 1.00% |
| Silicone oil | 40.00% | 40.00% | — | — | — |
| Sunflower oil | — | — | 40.00% | 40.00% | — |
| Mineral oil | — | — | — | — | 55.0% |
| Surfactant | 3.00% | 3.00% | 4.50% | 3.00% | 4.5% |
| Colorant | 0.05% | 0.05% | 0.05% | 0.05% | — |
| Water | 55.55% | 55.35% | 53.45% | 55.55% | 39.25% |

Examples 2-1 to 2-4 starting liquids were deposited into setting baths A and B and in all cases the beads formed were about 1 to 2 mm in diameter had a desired spherical shape and ruptured easily during gentle rubbing on the skin and left no residue. The beads formed from Examples 2-1 to 2-4 had greater bead strength compared to Examples 1-1 to 1-4.

About 50 to 60 beads prepared by depositing Examples 2-1 to 2-4 into both setting bath A and B were stored in a 2% calcium chloride solution. All beads were stable after one month storage in the calcium chloride solution. The beads remained individual and did not leak.

The beads prepared by depositing Example 2-5 (Comparative Example) into either setting bath A and B were soft but left a slight residue when rubbed on the skin.

### Examples 3-1 to 3-4

Starting liquids for beads made with CMCC and different types and levels of alginate.

| Components | 3-1 | 3-2 | 3-3 | 3-4 |
|---|---|---|---|---|
| AVICEL ® CL-611 | 1.20% | 1.20% | 1.20% | 1.20% |
| Alginate 1 | 0.20% | 0.40% | — | — |
| Alginate 2 | — | — | 0.20% | 0.40% |
| Silicone oil | 40.00% | 40.00% | 40.00% | 40.00% |
| Surfactant | 3.00% | 3.00% | 3.00% | 3.00% |
| Colorant | 0.05% | 0.05% | 0.05% | 0.05% |
| Water | 55.35% | 55.35% | 53.35% | 55.35% |

Examples 3-1 to 3-4 starting liquids were deposited into setting baths A and B and in all cases the beads formed were about 1 to 2 mm in diameter had a desired spherical shape and ruptured easily during gentle rubbing on the skin and left no residue.

### Examples 4-1 to 4-3

Starting liquids for beads made with colloidal microcrystalline cellulose and different levels of carrageenan.

| Components | 4-1 | 4-2 |
|---|---|---|
| AVICEL ® CL-611 | 1.20% | 1.20% |
| Iota carrageenan | 0.20% | 0.40% |
| Silicone oil | 40.00% | 40.00% |
| Surfactant | 3.00% | 3.00% |
| Colorant | 0.05% | 0.05% |
| Water | 55.35% | 55.35% |

Examples 3-1 to 3-4 starting liquids were deposited into setting baths A, B, C and D. In all cases, the beads formed had desirable shape and texture and disintegrated completely when rubbed on the skin. The stability of beads was evaluated after storage for 1 month in both deionized water and in a 2% salt solution. In the case of beads made using setting bath A or B the salt was calcium chloride. In the case of beads made using setting bath C or D the salt was potassium chloride. In the case of both Example 4-1 and 4-2, beads made using setting baths C and D were more stable during storage in water or the salt solution than those made using setting baths A and B.

### Examples 51 to 5-3

Alternative manufacturing process for beads containing colloidal microcrystalline cellulose and alginate.

| Components | 5-1 | 5-2 | 5-3 |
|---|---|---|---|
| AVICEL ® CL-611 | 1.20% | 1.20% | 1.20% |
| Alginate 3 | 0.20% | — | 0.40% |
| Alginate 4 | — | 0.40% | — |
| C12-15 Alkyl Benzoate | 40.00% | 40.00% | 40.00% |
| Surfactant | 3.00% | 3.00% | 3.00% |
| Colorant | 0.05% | 0.05% | 0.05% |
| Water | 55.55% | 55.55% | 53.55% |

The following procedure was used to prepare the beads

Phase 1: Alginate was dispersed in a premix of silicone oil, surfactant and colorant, and heated to 60° C. while stirring with a magnetic stirrer.

Phase 2: Colloidal microcrystalline cellulose was dispersed in deionized water with a Silverson rotor-stator mixer using high speed (8000 rpm) for 5 minutes and the dispersion was then heated to 60 C.

Phase 2 was added slowly to Phase 1 and homogenized using a pre-warmed rotor-stator mixer at high speed for 5 minutes. This emulsion was transferred to a syringe equipped with a pumping meter and was pumped at a rate of 60 ml per hour to a Var J1 Coaxial air-flow-driven single nozzle (manufactured by Nisco). The air pressure was adjusted to produce beads with diameters in the range of 0.5 to 1.0 mm. To form beads, the emulsion was delivered from the nozzle into a setting bath containing 1.2% calcium chloride dihydrate, 40% isopropanol and 58.8% deionized water. The bath was stirred gently and continuously using a magnetic stirrer. The beads were kept in the setting bath for 18 hours, then separated by sieving and washed by pouring deionized water slowly over beads in the sieve for 3 minutes. The beads were then placed in container of deionized water.

In all cases, the beads were approximately spherical and were stable for at least 1 week in deionized water at room temperature and at least 6 months in a 2% calcium chloride solution. The beads disintegrated completely when rubbed on the skin and had a pleasant skin-feel.

What is claimed is:

1. Beads comprising a carrier liquid and flocculated microcrystalline cellulose.

2. The beads of claim 1 further comprising a texture modifier.

3. Beads of claim 1 further comprising an oil.

4. A process for making the beads of claim 1 comprising the steps of a) dispersing colloidal microcrystalline cellulose in water to form a dispersion and b) flocculating the colloidal microcrystalline cellulose by adding said dispersion to a setting bath comprising a flocculating agent to form said beads.

5. A process for making the beads of claim 3 comprising the steps of a) dispersing colloidal microcrystalline cellulose in water to form a dispersion, b) homogenizing said dispersion with an oil phase comprising at least one oil to form an emulsion and c) flocculating the colloidal microcrystalline cellulose by adding said emulsion to a setting bath comprising a flocculating agent to form said beads.

6. A process of claim 4 wherein said flocculating agent is selected from a setting salt, an organic solvent, a pH modifier or a cationic material.

7. A composition comprising the beads of claim 1 wherein the composition disintegrates upon rubbing on the skin.

8. A cosmetic, pharmaceutical, neutraceutical, veterinary or food composition comprising the beads of claim 1.

9. Beads of claim 2 further comprising an oil.

10. A process for making the beads of claim 2 comprising the steps of a) dispersing colloidal microcrystalline cellulose in water to form a dispersion and b) flocculating the colloidal microcrystalline cellulose by adding said dispersion to a setting bath comprising a flocculating agent to form said beads.

11. A process of claim 5 wherein said flocculating agent is selected from a setting salt, an organic solvent, a pH modifier or a cationic material.

12. A composition comprising the beads of claim 2 wherein the composition disintegrates upon rubbing on the skin.

13. A composition comprising the beads of claim 3 wherein the composition disintegrates upon rubbing on the skin.
